(19) United States
(12) Patent Application Publication
(10) Pub. No.: US 2023/0295164 A1
(43) Pub. Date: Sep. 21, 2023

(54) COMPOUNDS HAVING ANTIBACTERIAL ACTIVITY

(71) Applicants: The Trustees of Princeton University, Princeton, NJ (US); PEKING UNIVERSITY SHENZHEN GRADUATE SCHOOL, Shenzhen (CN)

(72) Inventors: Zemer GITAI, Princeton, NJ (US); Hahn KIM, Princeton, NJ (US); James K. MARTIN, Princeton, NJ (US); Joseph P. SHEEHAN, Princeton, NJ (US); Connor CHAIN, Princeton, NJ (US); Yong HUANG, Shenzhen (CN); Shuo LI, Shenzhen (CN); Xueming CHEN, Shenzhen (CN); Chenran JIANG, Shenzhen (CN)

(21) Appl. No.: 18/014,032

(22) PCT Filed: Jul. 1, 2021
(86) PCT No.: PCT/US2021/040143

§ 371 Date: Dec. 30, 2022

Related U.S. Application Data
(60) Provisional application No. 63/047,612, filed on Jul. 2, 2020.

(51) Int. Cl. C07D 487/04; C07D 239/95; A61P 31/04

(52) U.S. Cl. CPC C07D 487/04; C07D 239/95; A61P 31/04

(57) ABSTRACT

In one aspect, compounds and associated pharmaceutical compositions are described herein for the treatment of various bacterial infections and/or other diseases. In some embodiments, for example, compounds of Formula (I) and/or salts thereof are described herein.

### RELATED APPLICATION DATA

The present application claims priority pursuant to Article 8 of the Patent Cooperation Treaty to U.S. Provisional Patent Application Ser. No. 63/047,612 filed Jul. 2, 2020 which is incorporated herein by reference in its entirety.

### STATEMENT OF GOVERNMENT RIGHTS

This invention was made with government support under Grant No. DP1AI124669 awarded by the National Institutes of Health (NIH). The government has certain rights in the invention.

### FIELD

The present invention relates to antibacterial compounds and modes of action associated with the compounds.

### BACKGROUND

The discovery of penicillin in 1929 ushered in the ‘Golden Age’ of antibiotic discovery and with it, over the next three decades, more than twenty unique classes of antibiotics. The discovery and development of these life-saving molecules has been in serious decline. Since the end of the ‘Golden Age’ in 1962 only two orally available antibiotics with completely novel targets, linezolid and a daptomycin, have been brought to the market. Declining rates of antibiotic discovery would be unalarming if it were not for evolution's perpetual offensive, constantly selecting antibiotic resistant bacteria through horizontal gene transfer and spontaneous mutation. In the United States alone, this manifests in a record 2 million antibiotic resistant infections, which annually kill 23,000 people. Moreover, such infections have been estimated to cost our health system as much as $35 billion annually. Other than better antibiotic stewardship, which has been shown to decrease the rate of hospital acquired infections, the only way to combat bacterial infections is to continuously develop antibiotics and other therapeutics with novel mechanisms of action (MOA), which have yet to slip into obsolescence.

### SUMMARY

In one aspect, compounds and associated pharmaceutical compositions are described herein for the treatment of various bacterial infections and/or other diseases. In some embodiments, for example, compounds of Formula (I) and/or salts thereof are provided:

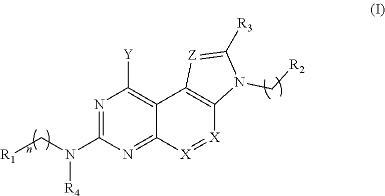

wherein R₁, R₃, R₄ and R₅ are independently selected from the group consisting of hydrogen, alkyl, alkenyl, heteroalkyl, cycloalkyl, heterocycloalkyl, aryl, heteroaryl, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of arylene-alkynyl, heteroarylene-alkynyl, arylene-alkenyl, heteroarylene-alkenyl, alkylnylene-alkyl, alkynylene-cycloalkyl, alkynylene-heterocycloalkyl, alkynylene-aryl, alkynylene-heteroaryl, alkenylene-aryl, alkenylene-heteroaryl, alkynylene-amine, alkynylene-protected amine, and alkynylene-alkylsilane; and
wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and
wherein Y is selected from the group consisting of OH and NR₁₂R₁₃, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure; and n is an integer from 0 to 5.

In another aspect, compounds of Formula (II) and/or salts thereof are provided:

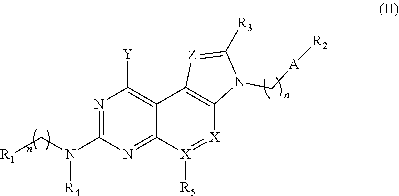

wherein R₁, R₃, R₄ and R₅ are independently selected from the group consisting of hydrogen, alkyl, alkenyl, heteroalkyl, cycloalkyl, heterocycloalkyl, aryl, heteroaryl, imine, cyanoimine, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of alkyl, cycloalkyl, heterocycloalkyl, alkynyl, alkenyl, alkylnylene-alkyl, alkynylene-cycloalkyl, alkynylene-heterocycloalkyl, alkynylene-aryl, alkynylene-heteroaryl, alkynylene-amine, alkynylene-protected amine, alkynylene-alkylsilane, fluoroalkyl, fluoro, bromo, B(OH)₂, nitro, cyano, and alkoxy; and
   wherein A is selected from the group consisting of aryl and heteroaryl; and
   wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and
   wherein Y is selected from the group consisting of OH, alkoxy, and NR₁₃R₁₄, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and
   n is an integer from 0 to 5.

In another aspect, compounds of Formula (III) and/or salts thereof are provided:

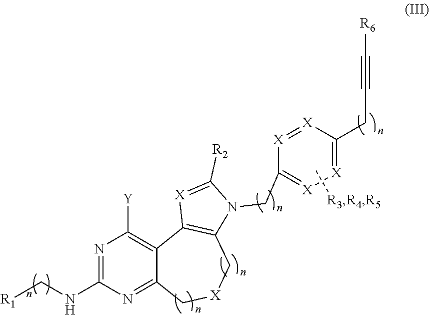

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and n is an integer from 0 to 5.

In another aspect, compounds of Formula (IV) and/or salts thereof are provided:

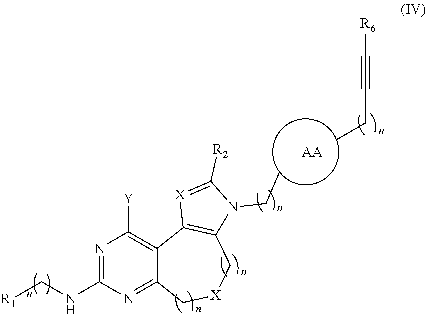

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and wherein AA is selected from the group consisting of arylene, heteroarylene, cycloalkylene, and heterocycloalkylene, and n is an integer from 0 to 5.

In another aspect, compounds of Formula (V) and/or salts thereof are provided:

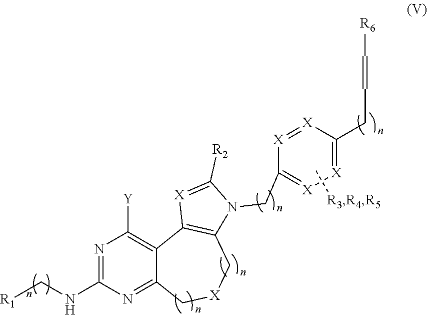

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and n is an integer from 0 to 5.

In another aspect, compounds of Formula (VI) and/or salts thereof are provided:

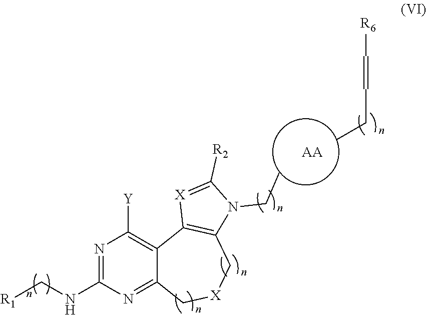

wherein R₁-R₆ are independently selected from the group consisting of hydrogen, alkyl cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and wherein AA is selected from the group consisting of arylene, heteroarylene, cycloalkylene, and heterocycloalkylene, and n is an integer from 0 to 5.

In a further aspect, compounds of Formula (VII) and/or salts thereof are provided:

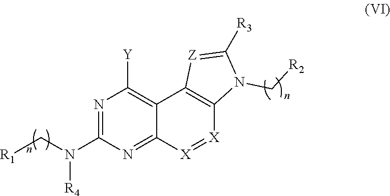

wherein R₁-R₄ are independently selected from the group consisting of hydrogen, alkyl, alkenyl, heteroalkyl, cycloalkyl, heterocycloalkyl, aryl, heteroaryl, alkyl-aryl, alkyl-heteroaryl, amide, sulfonamide, and urea, wherein the alkyl, alkenyl, heteroalkyl, cycloalkyl, heterocycloalkyl, aryl, heteroaryl, alkyl-aryl, alkyl-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, hydroxy, C(O)OR₅, and C(O)R₆, wherein R₅ is selected from the group consisting of hydrogen, alkyl and alkenyl and R₆ is selected from the group consisting of hydrogen, alkyl, alkenyl, heteroalkyl, cycloalkyl, heterocycloalkyl, aryl, heteroaryl and NR₇R₅, wherein R₇ and R₈ are independently selected from the group consisting of hydrogen, alkyl, alkenyl, aryl and heteroaryl; and
wherein X and Z are independently selected from the group consisting of C, N and O; and
wherein Y is selected from the group consisting of OH and NR₉R₁₀, wherein R₉ and R₁₀ are independently selected from the group consisting of hydrogen, alkyl, heteroalkyl, cycloalkyl, heterocycloalkyl, alkenyl, aryl, heteroaryl, amide, sulfonamide, urea and C(O)R₁₁ wherein R₁₁ is selected from the group consisting of hydrogen, alkyl, alkenyl, heteroalkyl, cycloalkyl, heterocycloalkyl, aryl and heteroaryl and wherein R₉ and R₁₀ may optionally form a ring structure;
and n is an integer from 0 to 5.

In another aspect, pharmaceutical compositions are described herein. A pharmaceutical composition comprises a compound selected from the group consisting of Formulas I-VII, wherein the compound is present in the pharmaceutical composition at a minimum inhibitory concentration (MIC) for treating a bacterial infection. In some embodiments, for example, the compound is present in the pharmaceutical composition in an amount of 0.0005 μm/ml to 200 μg/ml.

In another aspect, methods of treating bacterial infections are described herein. In some embodiments, a method comprises administering to a patient having a bacterial infection a therapeutically effective amount of one or more compounds of Formula(s) I-VII.

These and other embodiments are further described in the following detailed description.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents

The term “alkynyl” as used herein, alone or in combination, refers to a straight or branched chain hydrocarbon group having at least one carbon-carbon triple bond and optionally substituted with one or more substituents including, but not limited to, alkyl, cycloalkyl, heterocycloalkyl, aryl, heteroaryl, amine, and/or alkylsilane.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, oxygen and/or sulfur.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as nitrogen, oxygen or sulfur, alone or in combination, and wherein the ring system is optionally substituted with one or more ring substituents.

The term “heteroalkyl” as used herein, alone or in combination, refers to an alkyl moiety as defined above, having one or more carbon atoms in the chain, for example one, two or three carbon atoms, replaced with one or more heteroatoms, which may be the same or different, where the point of attachment to the remainder of the molecule is through a carbon atom of the heteroalkyl radical.

The term “alkoxy” as used herein, alone or in combination, refers to the moiety RO—, where R is alkyl or alkenyl defined above.

The term “halo” as used herein, alone or in combination, refers to elements of Group VIIA of the Periodic Table (halogens). Depending on chemical environment, halo can be in a neutral or anionic state. Halo, for example, includes fluoro, chloro, bromo, and iodo.

### I. Compounds and Pharmaceutical Compositions for Treating Bacterial Infections

Various compounds are described herein. As discussed above and further illustrated in the examples below, the compounds can exhibit antibacterial properties in some embodiments. The compounds can fall under any one of Formulas I-VII described above.

Pharmaceutical compositions employing such compounds exhibiting antibacterial activity are also provided. Compounds and/or salt(s) of Formulas I-VII can be individually administered in any amount consistent with treating bacterial infections. In some embodiments, one or more of the compounds are administered in an amount or concentration of 0.0005 μg/ml to 1 mg/ml. A compounds of any of Formulas I-VII can also be administered in an amount or concentration selected from Table I.

TABLE I
Amount of Compound of Formulas I-VII (μg/ml)
0.001-100
0.001-10
0.01-50
0.05-30
0.1-10

Additionally, compounds and/or salt(s) of Formulas I-VII. can be combined with any physiologically suitable carrier or excipient.

The amount or concentration of compounds of Formulas I-VII employed in pharmaceutical compositions described herein can be dependent on the identity and/or nature of the bacteria being treated. In some embodiments, bacteria of the infection treated with compounds described herein are gram positive. Alternatively, bacteria of the infection can be gram negative. Moreover, in some embodiments, two or more differing compounds selected from Formulas I-VII can be combined for treatment of bacterial infections. In some embodiments, some compounds of Formulas I-V are effective at treating the bacterial species and strains listed in Table II.

TABLE II
Bacterial Strains

| Species | Strain | Description |
|---|---|---|
| Clostridium difficile | ATCC BAA-1875 | Toxigenic |
| Propionibacterium acnes | ATCC 29399 | Human skin isolate |
| Acinetobacter baumannii | ATCC BAA- 1710 | Multi-drug resistant |
| Acinetobacter baumannii | ATCC 17978 | |
| Burkholderia cepacia | ATCC 25416 | |
| Citrobacter freundii | ATCC 8090 | |
| Enterococcus faecalis | ATCC 51575 | |
| Escherichia coli | NCTC 13461 | CTX-M betalactamase |
| | | positive |
| Escherichia coli | ATCC BAA-198 | |
| Haemophilus influenzae | ATCC 35056 | |
| Klebsiella pneumoniae | ATCC BAA-1705 | KPC carbapenemase |
| | | positive |
| Morganella morganii | ATCC 25830 | |
| Neisseria gonorrhoeae | CCUG 57598 | Cip-R, Cef-R |
| Proteus mirabilis | ATCC 29906 | |
| Pseudomonas aeruginosa | BCCM 27650 | Multi-drug resistant |
| Pseudomonas aeruginosa | PA14 | |
| Serratia marcescens | ATCC 13880 | |
| Stenotrophomonas | ATCC 13637 | |
| maltophila | | |
| Enterobacter cloacae | ATCC BAA-1143 | ESBL |
| Enterococcus faecium | ATCC BAA-2320 | Vancomycin resistant |
| Mycobacterium fortuitum | ATCC 110 | |
| Salmonella typhimurium | CMCC 50115 | |
| Staphylococcus aureus | NARSA NRS384 | Methicillin resistant |
| Staphylococcus aureus | NARSA VRS11b | Vancomycin resistant |
| Staphylococcus aureus | NARSA NRS17 | Intermediate |
| | | vancomycin resistance |
| Staphylococcus epidermidis | ATCC 51625 | Methicillin resistant |
| Streptococcus pneumoniae | NTU Hospital | Multi-drug resistant |
| | TM532 | |

In some embodiments, for example, one or more compounds falling under any one of Formulas I-VII can exhibit a MIC for a bacterial species/strain less than 10 μg/ml or less than 1 μg/ml. Additional MICs for a bacterial species/strain for compounds of falling under one or more of Formulas I-VII are provided in Table III.

TABLE III
MIC of Bacterial Species for Compounds
of Formulas I-VII (μg/ml)
0.0005-5
0.001-1
0.01-0.5
0.01-0.3
0.1-0.5

### II. Methods of Treating Bacterial Infections

In another aspect, methods of treating bacterial infections are described herein. In some embodiments, a method comprises administering to a patient having a bacterial infection a therapeutically effective amount of one or more compounds of Formula(s) I-VII. In some embodiments, a compound of any of Formulas I-VII is administered in an amount selected from Table I or Table III herein. In some embodiments, a combination of two or more compounds of any of Formulas I-VII can be employed in treating a bacterial infection. In some embodiments, bacterial infections treated with compounds described herein are selected from Table II.

These and other embodiments are further illustrated in the following non-limiting examples.

### Examples—Compounds Exhibiting Antibacterial Activity

Compounds falling under one or more of Formulas I-VII were prepared according to the following general reaction scheme. Common solvents were purified before use. All reagents were reagent grade and purified where necessary. Reactions were monitored by thin-layer chromatography (TLC) using Whatman precoated silica gel plates. Flash column chromatography was performed over ultrapure silica gel (200-400 mesh) from Merck. ¹H NMR spectra were recorded on a Bruker AVANCE 300 (300 MHz), 400 MHz or 500 MHz spectrometer. Multiplicities for ¹H NMR are designated as s=singlet, d=doublet, t=triplet, q=quartet, quint=quintet, sext=sextet, dd=doublet of doublets, dt=doublet of triplets, m=multiplet, and br=broad. Electrospray impact (ESI) mass spectra were recorded on ISQEC mass spectrometer.

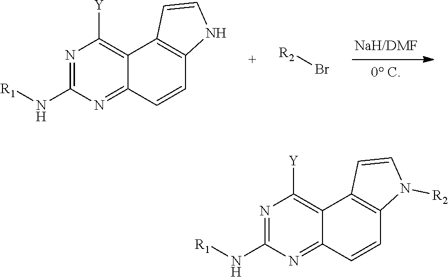

To a stirred solution of 7H-Pyrrolo[3, 2-f]quinazoline-1,3-diamine (1.0 mmol) in dry DMF (20 mL) was added NaH (1.2 mmol). The resulting reaction mixture was stirred at 0° C. for 0.5 h. Then corresponding bromide (1.5 mmol) was added. The reaction mixture was stirred at 0° C. for 1 h. The solvent was removed under reduced pressure and the residue was purified by column chromatography on silica gel, eluting with 10:1 DCM:MeOH containing 1% Et₃N to give the desired compound as a solid. The following specific compounds were synthesized according to the foregoing procedure.

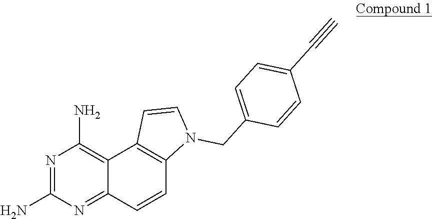 [SMILES: C#Cc1ccc(Cn2ccc3c4c(N)nc(N)nc4ccc32)cc1]

¹H NMR (500 MHz, DMSO-d₆) δ 7.71 (d, J=9.0 Hz, 1H), 7.62 (d, J=3.0 Hz, 1H), 7.41 (d, J=8.0 Hz, 2H), 7.15 (d, J=8.0 Hz, 2H), 7.12 (d, J=3.0 Hz, 1H), 7.03 (d, J=9.0 Hz, 1H), 6.78 (s, 2H), 5.89 (s, 2H), 5.53 (s, 2H), 4.14 (s, 1H). MS (ESI): [M+H⁺] 314.12.

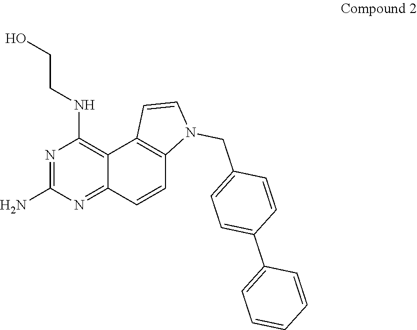 [SMILES: Nc1nc(NCCO)c2c(ccc3c2ccn3Cc2ccc(-c3ccccc3)cc2)n1]

¹H NMR (400 MHz, DMSO-d₆) δ 7.92 (d, J=9.0 Hz, 1H), 7.85 (d, J=3.2 Hz, 1H), 7.65-7.56 (m, 5H), 7.47-7.40 (m, 2H), 7.37-7.31 (m, 1H), 7.29-7.24 (m, 2H), 7.22-7.11 (m, 2H), 6.74 (s, 2H), 5.60 (s, 2H), 3.84-3.66 (m, 4H).

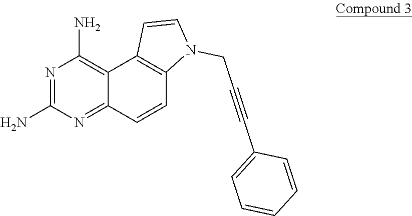 [SMILES: Nc1nc(N)c2c(ccc3c2ccn3CC#Cc2ccccc2)n1]

¹H NMR (300 MHz, DMSO-d₆) δ 7.88 (d, J=9.0 Hz, 1H), 7.58 (d, J=3.3 Hz, 1H), 7.46-7.33 (m, 5H), 7.17-7.06 (m, 2H), 6.70 (s, 2H), 5.72 (s, 2H), 5.43 (s, 2H).

MS(ESI): [M+H+]314.12.

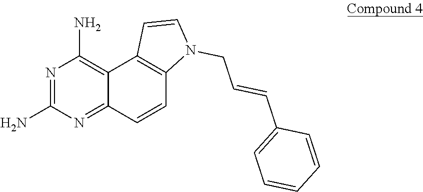 [SMILES: Nc1nc(N)c2c(ccc3c2ccn3C/C=C/c2ccccc2)n1]

¹H NMR (300 MHz, DMSO-d₆) δ 7.80 (d, J=9.0 Hz, 1H), 7.52 (d, J=3.0 Hz, 1H), 7.43-7.37 (m, 2H), 7.34-7.26 (m, 2H), 7.26-7.22 (m, 1H), 7.08 (d, J=3.0 Hz, 1H), 7.05 (d, J=9.0 Hz, 1H), 6.67 (s, 2H), 6.52-6.46 (m, 2H), 5.67 (s, 2H), 5.09-5.01 (m, 2H).

MS(ESI): [M+H+]316.15.

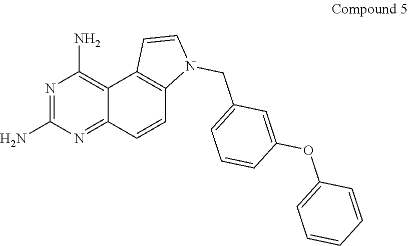 [SMILES: Nc1nc(N)c2c(ccc3c2ccn3Cc2cccc(Oc3ccccc3)c2)n1]

¹H NMR (300 MHz, DMSO-d₆) δ 7.71 (d, J=9.0 Hz, 1H), 7.60 (d, J=3.0 Hz, 1H), 7.39-7.25 (m, 3H), 7.13 (d, J=7.2 Hz, 1H), 7.08 (d, J=3.0 Hz, 1H), 7.02 (d, J=9.0 Hz, 1H), 6.97-6.89 (m, 3H), 6.87-6.80 (m, 2H), 6.68 (s, 2H), 5.69 (s, 2H), 5.50 (s, 2H).

MS(ESI): [M+H+] 382.12.

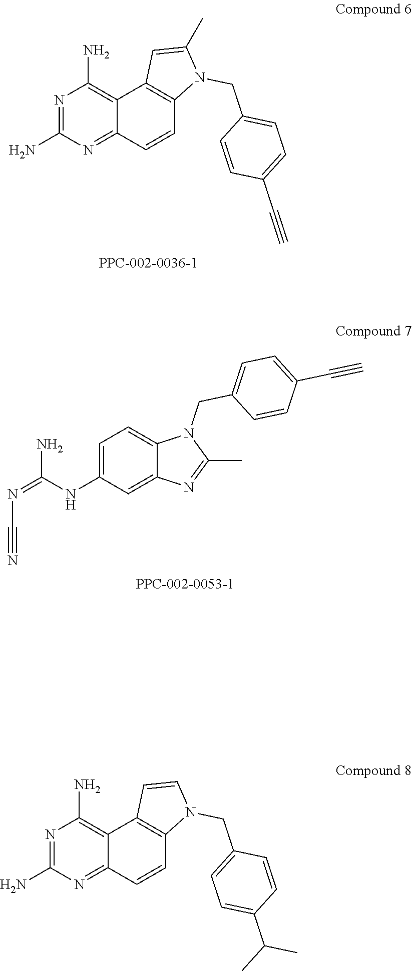 [SMILES: C#Cc1ccc(Cn2c(C)cc3c4c(N)nc(N)nc4ccc32)cc1 ; C#Cc1ccc(Cn2c(C)nc3cc(N/C(N)=N\C#N)ccc32)cc1 ; CC(C)c1ccc(Cn2ccc3c4c(N)nc(N)nc4ccc32)cc1]

¹H NMR (500 MHz, DMSO-d₆) δ 7.73 (d, J=9.0 Hz, 1H), 7.59 (d, J=3.0 Hz, 1H), 7.15 (d, J=8.0 Hz, 2H), 7.11 (d, J=8.0 Hz, 2H), 7.06 (d, J=3.0 Hz, 1H), 7.01 (d, J=8.9 Hz, 1H), 6.65 (s, 2H), 5.65 (s, 2H), 5.43 (s, 2H), 2.80 (p, J=7.0 Hz, 1H), 1.12 (d, J=6.9 Hz, 6H).

MS(ESI): [M+H+]332.42.

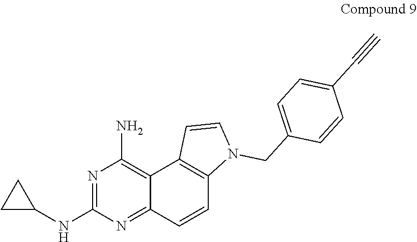 [SMILES: C#Cc1ccc(Cn2ccc3c4c(N)nc(NC5CC5)nc4ccc32)cc1]

¹H NMR (300 MHz, DMSO-d₆) δ 7.70 (d, J=9.0 Hz, 1H), 7.62 (d, J=3.0 Hz, 1H), 7.41 (d, J=8.2 Hz, 2H), 7.14 (d, J=8.2 Hz, 2H), 7.11 (d, J=3.0 Hz, 1H), 7.09 (d, J=9.0 Hz, 1H), 6.68 (s, 2H), 6.47-6.30 (m, 1H), 5.53 (s, 2H), 4.16 (s, 1H), 2.84-2.70 (m, 1H), 0.68-0.57 (m, 2H), 0.48-0.39 (m, 2H).

MS(ESI): [M+H+] 354.15.

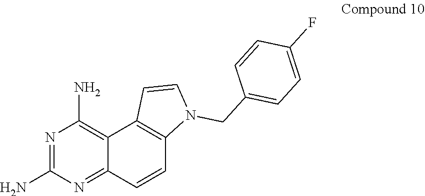 [SMILES: Nc1nc(N)c2c(ccc3c2ccn3Cc2ccc(F)cc2)n1]

¹H NMR (500 MHz, DMSO-d₆) δ 8.08 (d, J=9.0 Hz, 1H), 7.91 (d, J=3.0 Hz, 1H), 7.60 (s, 2H), 7.37 (d, J=3.0 Hz, 1H), 7.31-7.24 (m, 2H), 7.21 (d, J=9.0 Hz, 1H), 7.18-7.11 (m, 2H), 5.56 (s, 2H).

MS(ESI): [M+H+] 308.15.

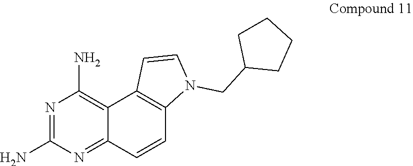 [SMILES: Nc1nc(N)c2c(ccc3c2ccn3CC2CCCC2)n1]

¹H NMR (500 MHz, DMSO-d₆) δ 7.78 (d, J=9.0 Hz, 1H), 7.47 (d, J=3.0 Hz, 1H), 7.05 (d, J=9.0 Hz, 1H), 7.00 (d, J=3.0 Hz, 1H), 6.68 (s, 2H), 5.69 (s, 2H), 4.13 (d, J=7.5 Hz, 2H), 2.42-2.29 (m, 1H), 1.66-1.58 (m, 2H), 1.58-1.52 (m, 2H), 1.51-1.44 (m, 2H), 1.28-1.20 (m, 2H).

MS(ESI): [M+H+] 282.24.

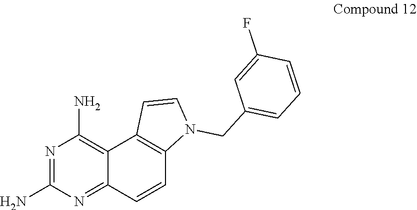 [SMILES: Nc1nc(N)c2c(ccc3c2ccn3Cc2cccc(F)c2)n1]

¹H NMR (400 MHz, DMSO-d₆) δ 7.92-7.82 (m, 1H), 7.80-7.72 (m, 1H), 7.39-7.31 (m, 1H), 7.25-7.18 (m, 1H), 7.14-7.05 (m, 2H), 7.04-6.96 (m, 2H), 5.55 (s, 2H).

MS(ESI): [M+H+] 308.06.

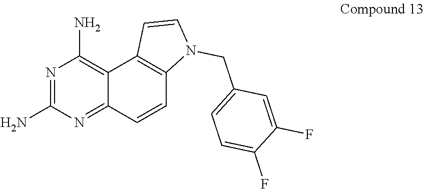 [SMILES: Nc1nc(N)c2c(ccc3c2ccn3Cc2ccc(F)c(F)c2)n1]

¹H NMR (400 MHz, DMSO-d₆) δ 7.75 (d, J=9.0 Hz, 1H), 7.63 (d, J=3.0 Hz, 1H), 7.41-7.34 (m, 1H), 7.34-7.26 (m, 1H), 7.11 (d, J=3.0 Hz, 1H), 7.03 (d, J=9.0 Hz, 1H), 7.02-6.99 (m, 1H), 6.72 (s, 2H), 5.73 (s, 2H), 5.49 (s, 2H).

MS(ESI): [M+H+] 326.10.

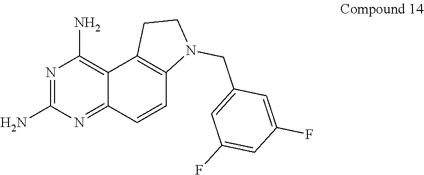 [SMILES: Nc1nc(N)c2c3c(ccc2n1)N(Cc1cc(F)cc(F)c1)CC3]

¹H NMR (400 MHz, DMSO-d₆) δ 7.76 (d, J=9.0 Hz, 1H), 7.66 (d, J=3.2 Hz, 1H), 7.18-7.09 (m, 2H), 7.05 (d, J=9.0 Hz, 1H), 6.90-6.84 (m, 2H), 6.78 (s, 2H), 5.81 (s, 2H), 5.54 (s, 2H).

MS(ESI): [M+H⁺] 326.10.

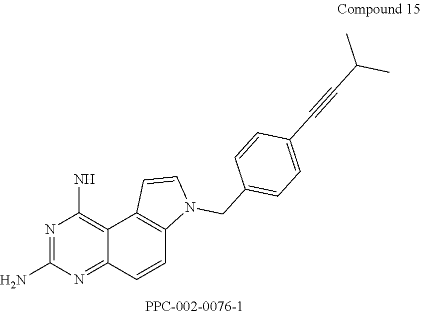 [SMILES: CC(C)C#Cc1ccc(Cn2ccc3c4c(N)nc(N)nc4ccc32)cc1]

¹H NMR (400 MHz, DMSO-d₆) δ 7.69 (d, J=9.0 Hz, 1H), 7.60 (d, J=3.2 Hz, 1H), 7.27 (d, J=8.0 Hz, 2H), 7.09 (d, J=8.0 Hz, 3H), 7.01 (d, J=9.0 Hz, 1H), 6.77 (s, 2H), 5.78 (s, 2H), 5.48 (s, 2H), 2.81-2.65 (m, 1H), 1.15 (d, J=6.8 Hz, 6H).

MS (ESI): [M+H⁺] 356.20

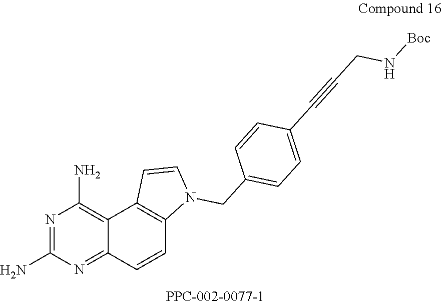 [SMILES: CC(C)(C)OC(=O)NCC#Cc1ccc(Cn2ccc3c4c(N)nc(N)nc4ccc32)cc1]

¹H NMR (500 MHz, DMSO-d₆) δ 7.70 (d, J=9.0 Hz, 1H), 7.61 (d, J=3.1 Hz, 1H), 7.34-7.29 (m, 2H), 7.29 (s, 1H), 7.18-7.05 (m, 3H), 7.01 (d, J=9.0 Hz, 1H), 6.79 (s, 2H), 5.79 (s, 2H), 5.49 (s, 2H), 3.91 (d, J=6.0 Hz, 2H), 1.35 (s, 9H).

MS (ESI): [M+H⁺] 443.53

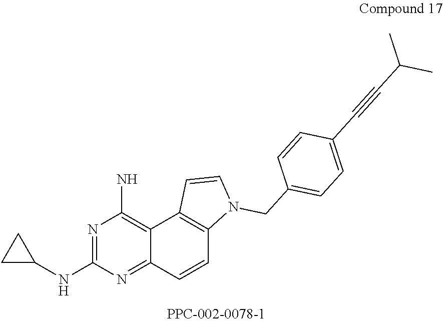 [SMILES: CC(C)C#Cc1ccc(Cn2ccc3c4c(N)nc(NC5CC5)nc4ccc32)cc1]

¹H NMR (400 MHz, DMSO-d₆) δ 7.68 (m, 1H), 7.64-7.59 (m, 1H), 7.34-7.26 (m, 2H), 7.15-7.06 (m, 4H), 6.67 (s, 2H), 6.37 (s, 1H), 5.51 (s, 2H), 2.83-2.74 (m, 1H), 1.18 (d, J=6.9 Hz, 6H), 0.63 (m, 2H), 0.51-0.37 (m, 2H).

MS (ESI): [M+H⁺] 396.50

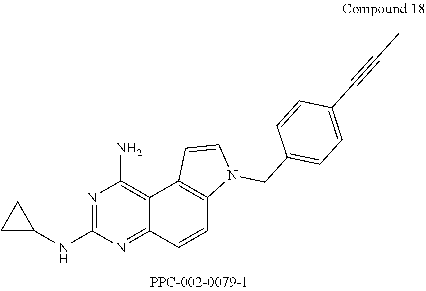 [SMILES: CC#Cc1ccc(Cn2ccc3c4c(N)nc(NC5CC5)nc4ccc32)cc1]

¹H NMR (400 MHz, DMSO-d₆) δ 7.73 (d, J=9.2 Hz, 1H), 7.69-7.60 (m, 1H), 7.32 (d, J=6.4 Hz, 2H), 7.15-7.07 (m, 4H), 5.51 (s, 2H), 2.88-2.72 (m, 1H), 0.86 (t, J=6.4 Hz, 2H), 0.64 (dd, J=6.4, 2.4 Hz, 2H).

MS (ESI): [M+H⁺] 368.45

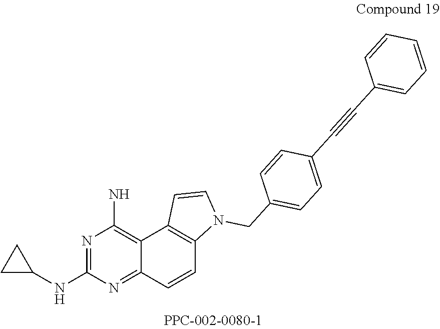 [SMILES: Nc1nc(NC2CC2)nc2ccc3c(ccn3Cc3ccc(C#Cc4ccccc4)cc3)c12]

¹H NMR (400 MHz, DMSO-d₆) δ 7.74 (d, J=8.8 Hz, 1H), 7.66 (d, J=3.2 Hz, 1H), 7.55-7.49 (m, 4H), 7.42 (d, J=3.2 Hz, 3H), 7.20 (d, J=8.0 Hz, 2H), 7.15-7.11 (m, 2H), 6.75 (s, 2H), 6.46 (s, 1H), 5.57 (s, 2H), 2.79 (m, 1H), 0.64 (m, 2H), 0.52-0.40 (m, 2H).

MS (ESI): [M+H⁺] 430.49

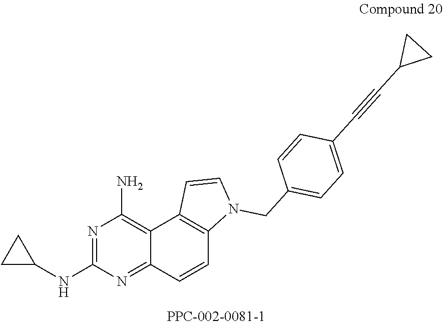 [SMILES: Nc1nc(NC2CC2)nc2ccc3c(ccn3Cc3ccc(C#CC4CC4)cc3)c12]

¹H NMR (400 MHz, DMSO-d₆) δ 7.71 (d, J=9.2 Hz, 1H), 7.61 (d, J=3.2 Hz, 1H), 7.33-7.26 (m, 2H), 7.16-7.06 (m, 4H), 6.69 (s, 2H), 6.40 (s, 1H), 5.50 (s, 2H), 2.79 (m, 1H), 1.51 (m, 1H), 0.91-0.81 (m, 4H), 0.73-0.59 (m, 4H)

MS (ESI): [M+H⁺] 394.19

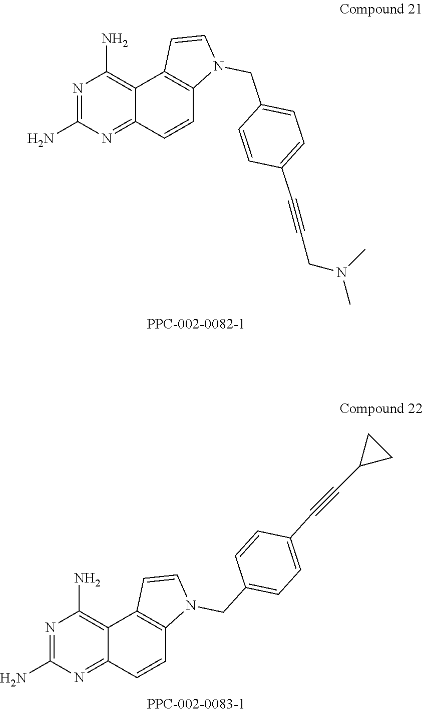

¹H NMR (400 MHz, DMSO-d₆) δ 7.70 (d, J=8.8 Hz, 1H), 7.61 (d, J=3.2 Hz, 1H), 7.29 (d, J=8.0 Hz, 2H), 7.17-7.08 (m, 3H), 7.02 (d, J=8.8 Hz, 1H), 6.73 (s, 2H), 5.76 (s, 2H), 5.50 (s, 2H), 1.50 (m, 1H), 0.91-0.79 (m, 2H), 0.72-0.64 (m, 2H).

MS (ESI): [M+H⁺] 354.46

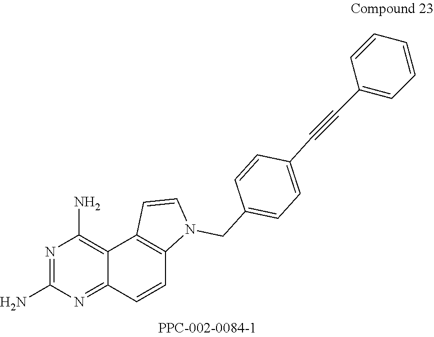

¹H NMR (400 MHz, DMSO-d₆) δ 7.80 (d, J=8.8 Hz, 1H), 7.71 (d, J=3.2 Hz, 1H), 7.57-7.46 (m, 4H), 7.42 (q, J=2.8 Hz, 3H), 7.25-7.16 (m, 3H), 7.08 (d, J=8.8 Hz, 1H), 6.13 (s, 2H), 5.58 (s, 2H).

MS (ESI): [M+H⁺] 390.41

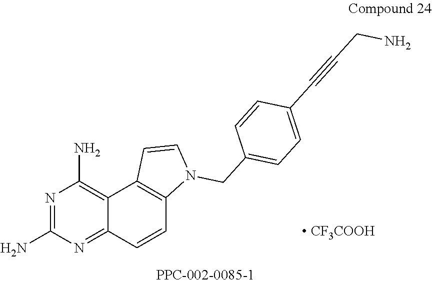 [SMILES: NCC#Cc1ccc(Cn2ccc3c4c(N)nc(N)nc4ccc32)cc1 ; "· CF3COOH"]

¹H NMR (400 MHz, DMSO-d₆) δ 8.06 (d, J=9.0 Hz, 1H), 7.93 (d, J=3.2 Hz, 1H), 7.67 (s, 2H), 7.47-7.37 (m, 3H), 7.22 (dd, J=9.0, 2.4 Hz, 3H), 5.64 (s, 2H), 3.98 (s, 2H).

MS (ESI): [M+H⁺] 343.38

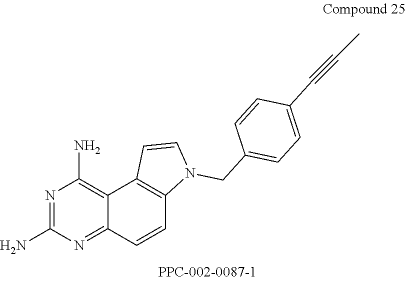

¹H NMR (400 MHz, DMSO-d₆) δ 7.75 (d, J=9.0 Hz, 1H), 7.65 (d, J=3.2 Hz, 1H), 7.37-7.26 (m, 2H), 7.17-7.10 (m, 3H), 7.04 (d, J=9.0 Hz, 1H), 6.88 (s, 2H), 5.93 (s, 2H), 5.51 (s, 2H), 2.00 (s, 3H).

MS (ESI): [M+H⁺] 328.37

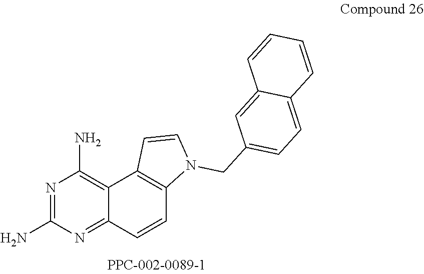 [SMILES: Nc1nc(N)c2c(ccc3c2ccn3Cc2ccc3ccccc3c2)n1]

¹H NMR (300 MHz, DMSO-d₆) δ 7.90-7.79 (m, 4H), 7.77 (d, J=3.2 Hz, 1H), 7.72 (s, 1H), 7.52-7.43 (m, 2H), 7.33 (dd, J=8.5, 1.8 Hz, 1H), 7.19 (d, J=3.2 Hz, 1H), 7.06 (d, J=9.0 Hz, 1H), 6.22 (s, 2H), 5.68 (s, 2H).

MS (ESI): [M+H⁺] 340.35

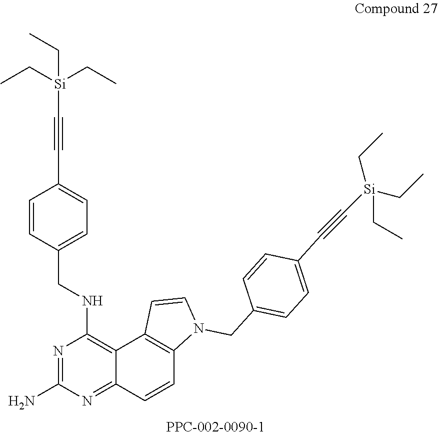

¹H NMR (500 MHz, DMSO-d₆) δ 7.70 (s, 1H), 7.65 (d, J=3.5 Hz, 1H), 7.41-7.34 (m, 6H), 7.31 (s, 1H), 7.23 (d, J=3.0 Hz, 1H), 7.14 (d, J=8.0 Hz, 2H), 5.86 (s, 2H), 5.51 (s, 2H), 4.83 (d, J=6.0 Hz, 2H), 0.95 (m, 18H), 0.59 (m, 12H).

MS (ESI): [M+H⁺] 657.03

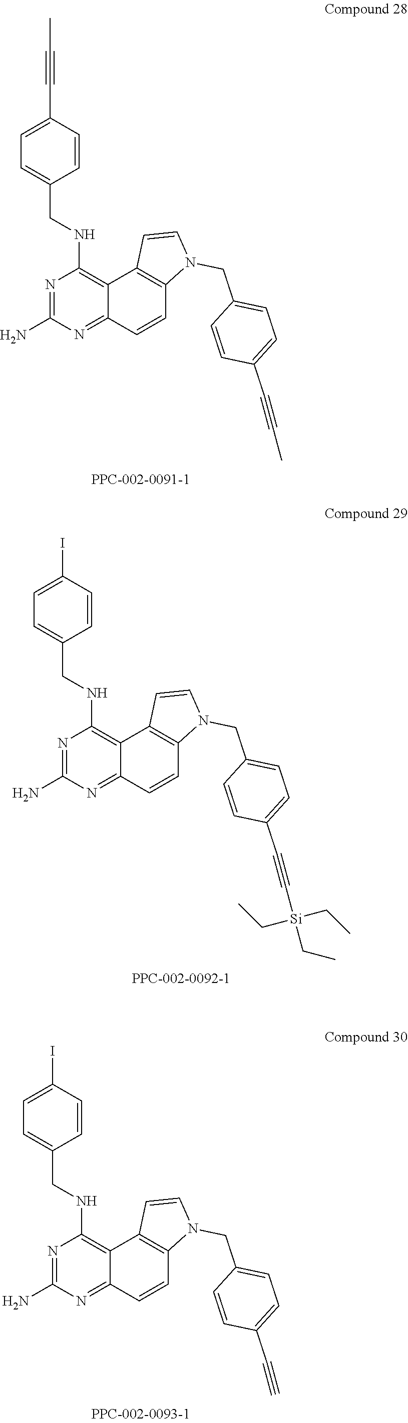 [SMILES: CC#Cc1ccc(CNc2nc(N)nc3ccc4c(ccn4Cc4ccc(C#CC)cc4)c23)cc1 ; CC[Si](C#Cc1ccc(Cn2ccc3c4c(NCc5ccc(I)cc5)nc(N)nc4ccc32)cc1)(CC)CC ; C#Cc1ccc(Cn2ccc3c4c(NCc5ccc(I)cc5)nc(N)nc4ccc32)cc1]

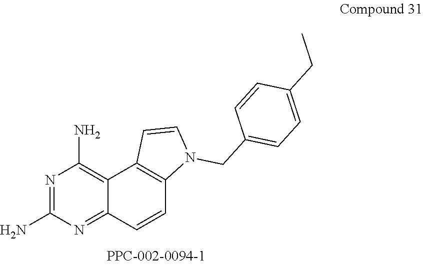 [SMILES: CCc1ccc(Cn2ccc3c4c(N)nc(N)nc4ccc32)cc1]

¹H NMR (400 MHz, DMSO-d₆) δ 7.79 (d, J=9.0 Hz, 1H), 7.65 (d, J=3.2 Hz, 1H), 7.13 (t, J=3.2 Hz, 5H), 7.05 (d, J=9.0 Hz, 1H), 6.92 (s, 2H), 5.98 (s, 2H), 5.46 (s, 2H), 2.55 (m, 2H), 1.12 (t, J=7.6 Hz, 3H).

MS (ESI): [M+H⁺] 318.36

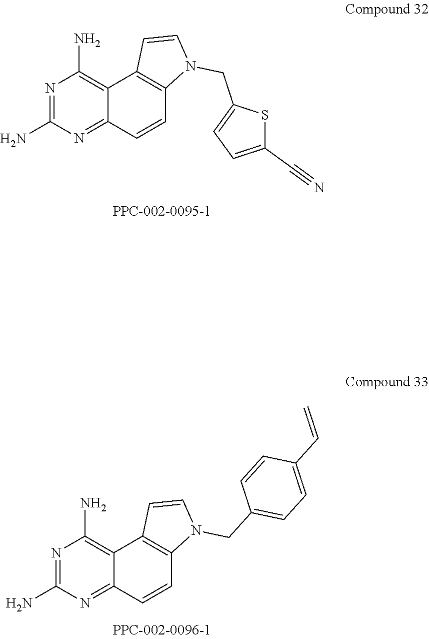 [SMILES: N#Cc1ccc(Cn2ccc3c4c(N)nc(N)nc4ccc32)s1 ; C=Cc1ccc(Cn2ccc3c4c(N)nc(N)nc4ccc32)cc1]

¹H NMR (500 MHz, DMSO-d₆) δ 7.71 (d, J=9.0 Hz, 1H), 7.60 (d, J=3.0 Hz, 1H), 7.41-7.34 (m, 2H), 7.13 (d, J=8.2 Hz, 2H), 7.07 (d, J=3.0 Hz, 1H), 7.00 (d, J=9.0 Hz, 1H), 6.74 (s, 2H), 6.65 (dd, J=17.6, 11.0 Hz, 1H), 5.81-5.68 (m, 3H), 5.46 (s, 2H), 5.19 (dd, J=10.9, 1.0 Hz, 1H).

MS (ESI): [M+H⁺] 316.40

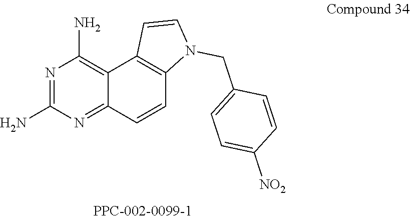 [SMILES: Nc1nc(N)c2c(ccc3c2ccn3Cc2ccc([N+](=O)[O-])cc2)n1]

¹H NMR (400 MHz, DMSO-d₆) δ 8.22-8.17 (m, 2H), 7.96 (d, J=9.0 Hz, 1H), 7.91 (d, J=3.2 Hz, 1H), 7.43-7.35 (m, 3H), 7.19 (d, J=9.0 Hz, 1H), 7.12 (s, 2H), 5.76 (s, 2H).

MS (ESI): [M+H+] 335.08

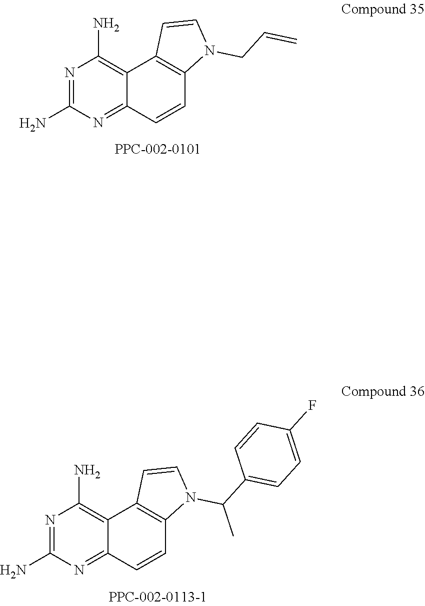 [SMILES: C=CCn1ccc2c3c(N)nc(N)nc3ccc21 ; CC(c1ccc(F)cc1)n1ccc2c3c(N)nc(N)nc3ccc21]

¹H NMR (400 MHz, DMSO-d₆) δ 7.80 (d, J=3.2 Hz, 1H), 7.74 (d, J=8.8 Hz, 1H), 7.33-7.23 (m, 2H), 7.19-7.07 (m, 3H), 7.01 (d, J=8.8 Hz, 1H), 6.80 (s, 2H), 5.95 (q, J=6.8 Hz, 1H), 5.81 (s, 2H), 1.90 (d, J=6.8 Hz, 3H).

MS (ESI): [M+H⁺] 322.32

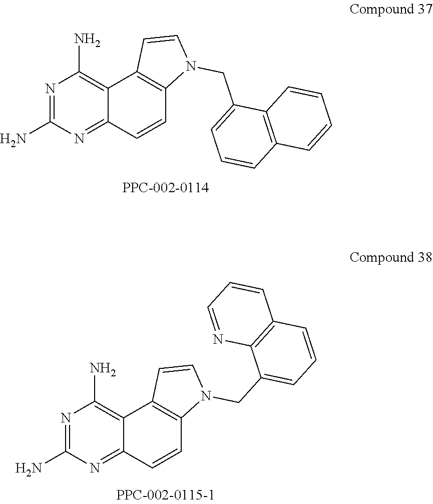 [SMILES: Nc1nc(N)c2c(ccc3c2ccn3Cc2cccc3ccccc23)n1 ; Nc1nc(N)c2c(ccc3c2ccn3Cc2cccc3cccnc23)n1]

¹H NMR (400 MHz, DMSO-d₆) δ 9.07 (dd, J=4.0, 2.0 Hz, 1H), 8.43 (dd, J=8.4, 2.0 Hz, 1H), 7.92 (d, J=8.0 Hz, 1H), 7.80 (d, J=8.8 Hz, 1H), 7.73 (d, J=3.2 Hz, 1H), 7.65 (dd, J=8.4, 4.4 Hz, 1H), 7.47 (t, J=8.0 Hz, 1H), 7.16 (d, J=3.2 Hz, 1H), 7.03 (dd, J=8.4, 4.4 Hz, 2H), 6.92 (s, 2H), 6.15 (s, 2H), 5.95 (s, 2H).

MS (ESI): [M+H⁺] 341.33

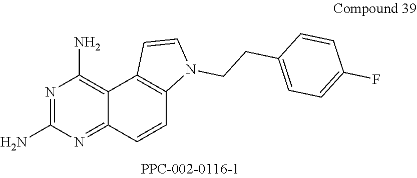 [SMILES: Nc1nc(N)c2c(ccc3c2ccn3CCc2ccc(F)cc2)n1]

¹H NMR (400 MHz, DMSO-d₆) δ 7.78 (d, J=8.8 Hz, 1H), 7.37 (d, J=3.2 Hz, 1H), 7.25-7.14 (m, 2H), 7.12-6.94 (m, 4H), 6.73 (s, 2H), 5.79 (s, 2H), 4.47 (t, J=7.2 Hz, 2H), 3.08 (t, J=7.2 Hz, 2H).

MS (ESI): [M+H⁺] 341.33

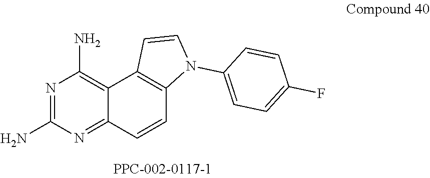 [SMILES: Nc1nc(N)c2c(ccc3c2ccn3-c2ccc(F)cc2)n1]

¹H NMR (300 MHz, Methanol-d₄) δ 7.78 (dd, J=9.1, 0.9 Hz, 1H), 7.66 (d, J=3.3 Hz, 1H), 7.62-7.55 (m, 2H), 7.41-7.30 (m, 2H), 7.25-7.18 (m, 2H).

MS (ESI): [M+H⁺] 294.30

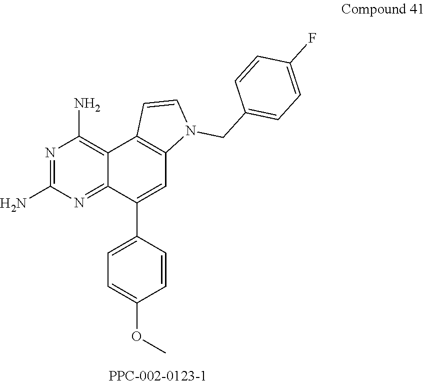 [SMILES: COc1ccc(-c2cc3c(ccn3Cc3ccc(F)cc3)c3c(N)nc(N)nc23)cc1]

¹H NMR (400 MHz, DMSO-d₆) δ 7.74 (s, 1H), 7.65 (d, J=3.2 Hz, 1H), 7.55-7.46 (m, 2H), 7.30-7.20 (m, 2H), 7.20-7.09 (m, 3H), 7.01-6.94 (m, 2H), 6.83 (s, 2H), 5.71 (s, 2H), 5.54 (s, 2H), 3.80 (s, 3H).

MS (ESI): [M+H⁺] 414.48

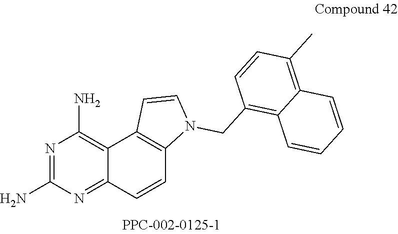 [SMILES: Cc1ccc(Cn2ccc3c4c(N)nc(N)nc4ccc32)c2ccccc12]

¹H NMR (400 MHz, DMSO-d₆) δ 8.22-8.13 (m, 1H), 8.12-8.03 (m, 1H), 7.72 (d, J=9.2 Hz, 1H), 7.66-7.57 (m, 2H), 7.48 (d, J=3.2 Hz, 1H), 7.24 (d, J=7.2 Hz, 1H), 7.11 (d, J=3.2 Hz, 1H), 7.02 (d, J=9.2 Hz, 1H), 6.70 (s, 2H), 6.68 (s, 1H), 5.97 (s, 2H), 5.70 (s, 2H), 2.62 (s, 3H).

MS (ESI): [M+H⁺] 354.44

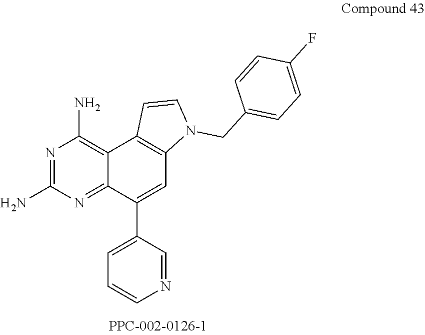 [SMILES: Nc1nc(N)c2c(n1)c(-c1cccnc1)cc1c2ccn1Cc1ccc(F)cc1]

¹H NMR (400 MHz, Methanol-d₄) δ 8.70 (dd, J=2.2, 0.8 Hz, 1H), 8.57 (dd, J=4.8, 1.6 Hz, 1H), 8.04 (dt, J=8.0, 2.0 Hz, 1H), 7.83 (d, J=0.8 Hz, 1H), 7.66 (d, J=3.2 Hz, 1H), 7.56 (m, 1H), 7.29-7.18 (m, 2H), 7.13 (dd, J=3.2, 0.8 Hz, 1H), 7.10-7.00 (m, 2H), 5.56 (s, 2H).

MS (ESI): [M+H⁺] 385.41

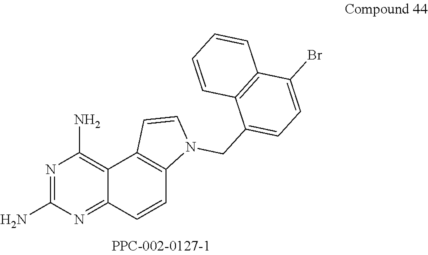 [SMILES: Nc1nc(N)c2c(ccc3c2ccn3Cc2ccc(Br)c3ccccc23)n1]

¹H NMR (400 MHz, DMSO-d₆) δ 8.25 (m, 2H), 7.79-7.70 (m, 4H), 7.55 (d, J=3.1 Hz, 1H), 7.17 (d, J=3.1 Hz, 1H), 7.03 (d, J=8.9 Hz, 1H), 6.76 (s, 2H), 6.54 (d, J=7.7 Hz, 1H), 6.04 (s, 2H), 5.76 (s, 2H).

MS (ESI): [M+H⁺] 418.31

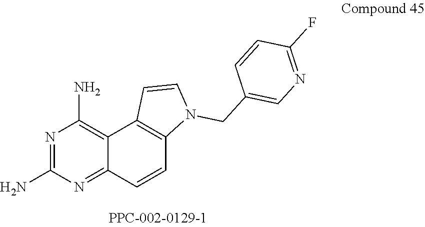 [SMILES: Nc1nc(N)c2c(ccc3c2ccn3Cc2ccc(F)nc2)n1]

¹H NMR (400 MHz, DMSO-d₆) δ 8.24 (d, J=2.4 Hz, 1H), 7.99 (d, J=8.9 Hz, 1H), 7.82 (t, J=5.5 Hz, 2H), 7.47 (s, 2H), 7.25 (d, J=3.2 Hz, 1H), 7.15 (dd, J=8.9, 2.4 Hz, 2H), 6.58 (s, 2H), 5.59 (s, 2H).

MS (ESI): [M+H⁺] 309.36

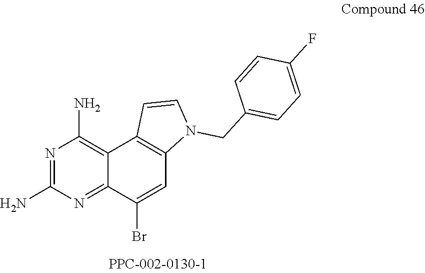 [SMILES: Nc1nc(N)c2c(n1)c(Br)cc1c2ccn1Cc1ccc(F)cc1]

¹H NMR (400 MHz, DMSO-d₆) δ 8.21 (s, 1H), 7.66 (d, J=3.2 Hz, 1H), 7.29-7.20 (m, 2H), 7.20-7.09 (m, 3H), 6.85 (s, 2H), 5.94 (s, 2H), 5.51 (s, 2H).

MS (ESI): [M+H⁺] 386.21

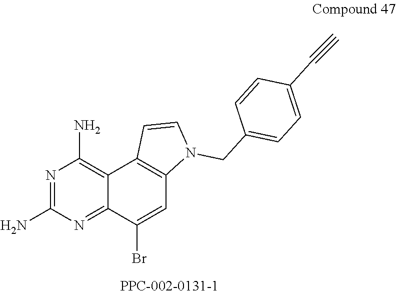 [SMILES: C#Cc1ccc(Cn2ccc3c4c(N)nc(N)nc4c(Br)cc32)cc1]

¹H NMR (400 MHz, Chloroform-d+MeOD) δ 7.82 (d, J=1.2 Hz, 1H), 7.30 (d, J=8.0 Hz, 2H), 7.25-7.23 (m, 1H), 6.90 (d, J=8.0 Hz, 2H), 6.72 (d, J=3.2 Hz, 1H), 5.29 (s, 2H), 3.03 (s, 1H).

MS (ESI): [M+H⁺] 393.25

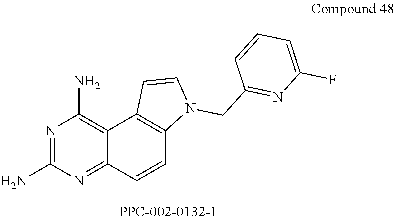 [SMILES: Nc1nc(N)c2c(ccc3c2ccn3Cc2cccc(F)n2)n1]

¹H NMR (400 MHz, DMSO-d₆) δ 8.00-7.86 (m, 2H), 7.77 (d, J=3.0 Hz, 1H), 7.65 (s, 1H), 7.29 (d, J=3.0 Hz, 1H), 7.15 (d, J=8.8 Hz, 1H), 7.09 (dd, J=8.0, 2.4 Hz, 1H), 6.95 (dd, J=8.0, 2.4 Hz, 1H), 6.76 (s, 2H), 5.63 (s, 2H).

MS (ESI): [M+H⁺] 309.33

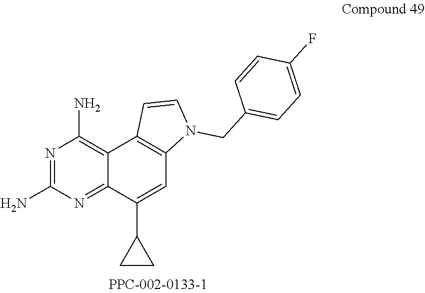 [SMILES: Nc1nc(N)c2c(n1)c(C1CC1)cc1c2ccn1Cc1ccc(F)cc1]

¹H NMR (400 MHz, Methanol-d₄) δ 7.59 (d, J=4.4 Hz, 2H), 7.27-7.15 (m, 2H), 7.13-6.98 (m, 3H), 5.53 (s, 2H), 2.20 (m, 1H), 1.15-1.03 (m, 2H), 0.76-0.65 (m, 2H).

MS (ESI): [M+H⁺] 348.45

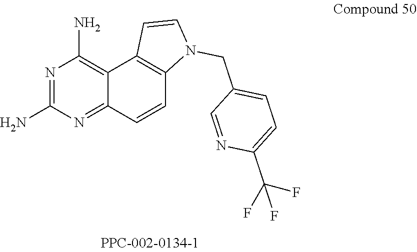 [SMILES: Nc1nc(N)c2c(ccc3c2ccn3Cc2ccc(C(F)(F)F)nc2)n1]

¹H NMR (400 MHz, Chloroform-d+MeOD) δ 8.35 (d, J=2.0 Hz, 1H), 7.45 (m 2H), 7.35 (dd, J=8.4, 2.0 Hz, 1H), 7.28 (s, 1H), 7.03 (d, J=8.4 Hz, 1H), 6.77 (d, J=3.2 Hz, 1H), 5.42 (s, 2H).

MS (ESI): [M+H⁺] 359.30

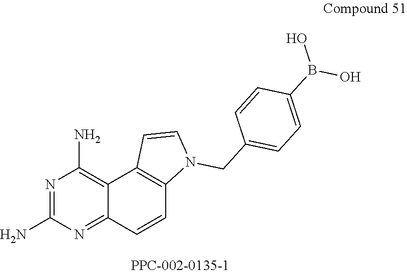 [SMILES: Nc1nc(N)c2c(ccc3c2ccn3Cc2ccc(B(O)O)cc2)n1]

¹H NMR (400 MHz, DMSO-d₆) δ 8.03 (m, 3H), 7.92 (d, J=3.2 Hz, 1H), 7.71 (d, J=7.6 Hz, 2H), 7.45 (s, 2H), 7.38 (d, J=3.2 Hz, 1H), 7.21 (d, J=8.8 Hz, 1H), 7.15 (d, J=7.6 Hz, 2H), 5.59 (s, 2H).

MS (ESI): [M+H⁺] 334.17

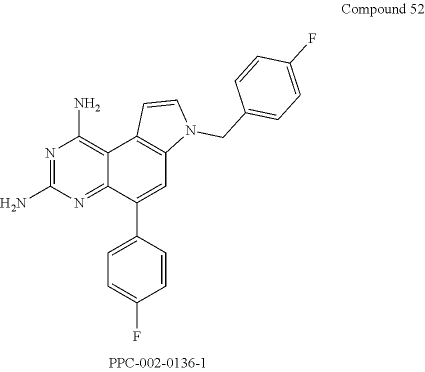 [SMILES: Nc1nc(N)c2c(n1)c(-c1ccc(F)cc1)cc1c2ccn1Cc1ccc(F)cc1]

¹H NMR (400 MHz, Methanol-d₄) δ 7.79 (d, J=0.8 Hz, 1H), 7.69 (d, J=3.2 Hz, 1H), 7.56-7.44 (m, 2H), 7.30-7.19 (m, 4H), 7.16 (dd, J=3.2, 0.8 Hz, 1H), 7.10-7.01 (m, 2H), 5.55 (s, 2H).

MS (ESI): [M+H⁺] 402.40

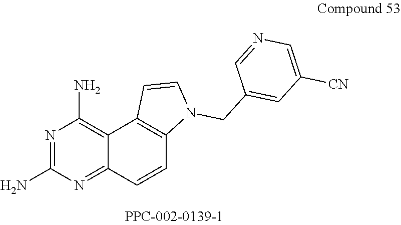 [SMILES: N#Cc1cncc(Cn2ccc3c4c(N)nc(N)nc4ccc32)c1]

¹H NMR (400 MHz, DMSO-d₆) δ 8.94 (d, J=2.0 Hz, 1H), 8.76 (d, J=2.0 Hz, 1H), 8.18 (d, J=2.0 Hz, 1H), 7.94 (d, J=9.0 Hz, 1H), 7.78 (d, J=3.2 Hz, 1H), 7.23 (d, J=3.2 Hz, 1H), 7.11 (d, J=9.0 Hz, 1H), 6.28 (s, 2H), 5.64 (s, 2H).

MS (ESI): [M+H⁺] 316.31

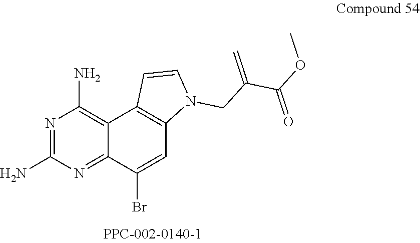 [SMILES: C=C(Cn1ccc2c3c(N)nc(N)nc3c(Br)cc21)C(=O)OC]

¹H NMR (400 MHz, Methanol-d₄) δ 8.11 (d, J=1.2 Hz, 1H), 7.45 (d, J=3.2 Hz, 1H), 6.96 (dd, J=3.2, 0.8 Hz, 1H), 6.27 (d, J=1.0 Hz, 1H), 5.31 (d, J=1.0 Hz, 1H), 5.14 (t, J=1.6 Hz, 2H), 3.79 (s, 3H).

MS (ESI): [M+H⁺] 376.22

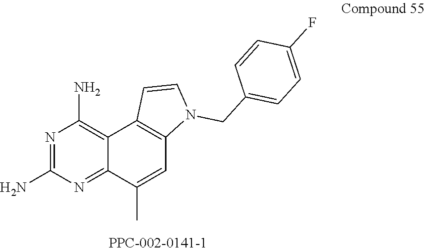 [SMILES: Cc1cc2c(ccn2Cc2ccc(F)cc2)c2c(N)nc(N)nc12]

¹H NMR (400 MHz, Methanol-d₄) δ 7.60 (d, J=3.2 Hz, 1H), 7.41 (t, J=3.2 Hz, 1H), 7.19-7.14 (m, 2H), 7.04 (dd, J=8.8, 3.2 Hz, 2H), 6.95 (t, J=3.2 Hz, 1H), 5.44 (d, J=3.2 Hz, 2H), 2.52 (d, J=3.2 Hz, 3H).

MS (ESI): [M+H⁺] 322.34

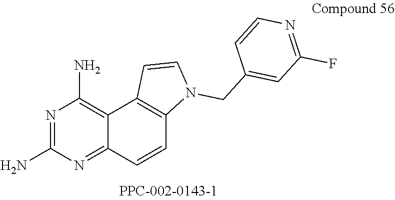 [SMILES: Nc1nc(N)c2c(ccc3c2ccn3Cc2ccnc(F)c2)n1]

¹H NMR (400 MHz, DMSO-d₆) δ 8.16 (d, J=5.2 Hz, 1H), 7.71 (d, J=9.0 Hz, 1H), 7.66 (d, J=3.2 Hz, 1H), 7.18 (d, J=3.2 Hz, 1H), 7.05 (d, J=9.0 Hz, 1H), 7.00-6.98 (m, 1H), 6.84 (d, J=1.5 Hz, 1H), 6.79 (s, 2H), 5.80 (s, 2H), 5.64 (s, 2H).

MS (ESI): [M+H⁺] 309.36

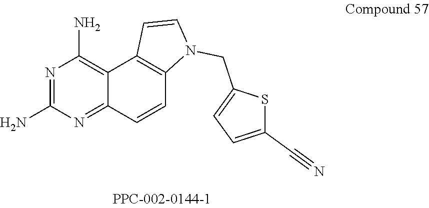 [SMILES: N#Cc1ccc(Cn2ccc3c4c(N)nc(N)nc4ccc32)s1]

¹H NMR (400 MHz, DMSO-d₆) δ 7.91-7.79 (m, 3H), 7.63 (d, J=3.2 Hz, 1H), 7.11 (d, J=3.2 Hz, 1H), 7.05 (d, J=8.8 Hz, 1H), 6.74 (s, 2H), 5.77 (s, 2H), 5.51 (s, 2H).

MS (ESI): [M+H⁺] 321.38

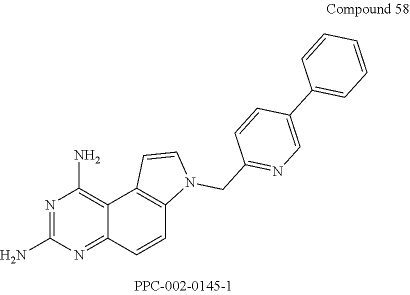 [SMILES: Nc1nc(N)c2c(ccc3c2ccn3Cc2ccc(-c3ccccc3)cn2)n1]

¹H NMR (400 MHz, DMSO-d₆) δ 8.85 (d, J=2.4 Hz, 1H), 8.01 (dd, J=8.4, 2.4 Hz, 1H), 7.78 (d, J=9.2 Hz, 1H), 7.74-7.63 (m, 3H), 7.48 (t, J=7.2 Hz, 2H), 7.41 (t, J=7.2 Hz, 1H), 7.15 (d, J=3.2 Hz, 1H), 7.05 (d, J=8.4 Hz, 2H), 6.82 (s, 2H), 5.83 (s, 2H), 5.65 (s, 2H).

MS (ESI): [M+H⁺] 367.41

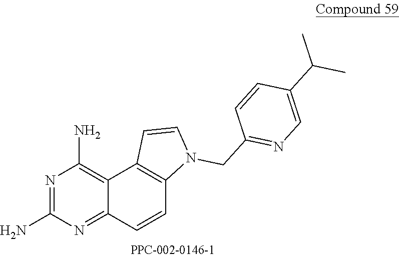 [SMILES: CC(C)c1ccc(Cn2ccc3c4c(N)nc(N)nc4ccc32)nc1]

¹H NMR (400 MHz, DMSO-d₆) δ 8.42 (d, J=2.4 Hz, 1H), 7.77 (d, J=8.8 Hz, 1H), 7.68-7.54 (m, 2H), 7.13 (d, J=3.2 Hz, 1H), 7.04 (d, J=8.8 Hz, 1H), 6.92 (d, J=8.0 Hz, 1H), 6.87 (s, 2H), 5.91 (s, 2H), 5.55 (s, 2H), 2.88 (m, 1H), 1.17 (d, J=6.8 Hz, 6H).

MS (ESI): [M+H⁺] 333.40

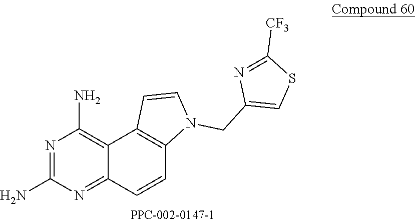 [SMILES: Nc1nc(N)c2c(ccc3c2ccn3Cc2csc(C(F)(F)F)n2)n1]

¹H NMR (400 MHz, DMSO-d₆) δ 7.92-7.82 (m, 2H), 7.62 (d, J=3.2 Hz, 1H), 7.13 (d, J=3.2 Hz, 1H), 7.07 (d, J=9.2 Hz, 1H), 6.83 (s, 2H), 5.87 (s, 2H), 5.71 (s, 2H).

MS (ESI): [M+H⁺] 365.33

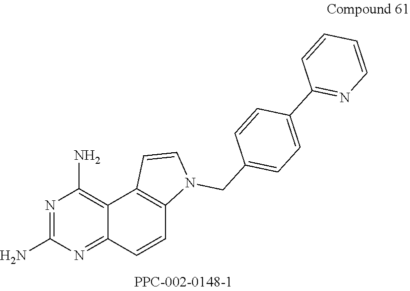 [SMILES: Nc1nc(N)c2c(ccc3c2ccn3Cc2ccc(-c3ccccn3)cc2)n1]

¹H NMR (400 MHz, DMSO-d₆) δ 8.63 (dt, J=4.8, 1.6 Hz, 1H), 8.06-7.98 (m, 2H), 7.94-7.81 (m, 2H), 7.78 (d, J=9.0 Hz, 1H), 7.69 (d, J=3.2 Hz, 1H), 7.35-7.25 (m, 3H), 7.15 (d, J=3.2 Hz, 1H), 7.05 (d, J=9.0 Hz, 1H), 6.85 (s, 2H), 5.93-5.80 (m, 2H), 5.58 (s, 2H).

MS (ESI): [M+H⁺] 367.41

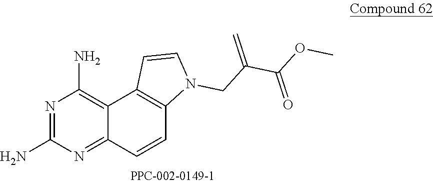 [SMILES: C=C(Cn1ccc2c3c(N)nc(N)nc3ccc21)C(=O)OC]

¹H NMR (400 MHz, DMSO-d₆) δ 7.71 (d, J=8.8 Hz, 1H), 7.45 (d, J=3.2 Hz, 1H), 7.14-6.99 (m, 2H), 6.66 (s, 2H), 6.15 (s, 1H), 5.67 (s, 2H), 5.24 (s, 1H), 5.14 (s, 2H), 3.73 (s, 3H).

MS (ESI): [M+H⁺] 298.11

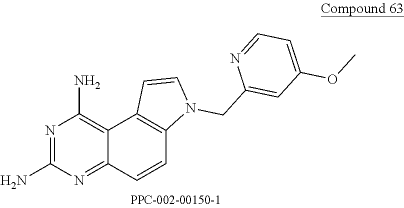 [SMILES: COc1ccnc(Cn2ccc3c4c(N)nc(N)nc4ccc32)c1]

¹H NMR (400 MHz, DMSO-d₆) δ 8.47-8.25 (m, 1H), 7.91-7.55 (m, 2H), 7.01 (m, 5H), 6.59 (s, 1H), 6.04 (s, 2H), 5.52 (s, 2H), 3.73 (s, 3H).

MS (ESI): [M+H+] 321.20.

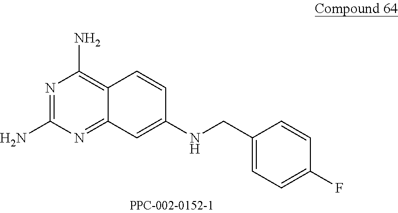 [SMILES: Nc1nc(N)c2ccc(NCc3ccc(F)cc3)cc2n1]

¹H NMR (400 MHz, DMSO-d₆) δ 7.83 (s, 2H), 7.51-7.38 (m, 2H), 7.19-7.11 (m, 4H), 7.08 (s, 1H), 6.55 (s, 2H), 6.29 (t, J=6.4 Hz, 1H), 4.29 (d, J=6.0 Hz, 2H).

MS (ESI): [M+H⁺] 284.30

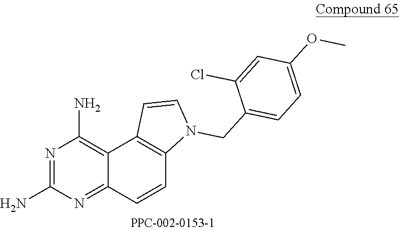 [SMILES: COc1ccc(Cn2ccc3c4c(N)nc(N)nc4ccc32)c(Cl)c1]

¹H NMR (400 MHz, DMSO-d₆) δ 7.70 (d, J=9.2 Hz, 1H), 7.51 (d, J=3.2 Hz, 1H), 7.11 (dd, J=8.0, 2.8 Hz, 2H), 7.05 (d, J=9.2 Hz, 1H), 6.84 (dd, J=8.4, 2.4 Hz, 1H), 6.76 (s, 2H), 6.73 (d, J=8.4 Hz, 1H), 5.78 (s, 2H), 5.50 (s, 2H), 3.74 (s, 3H).

MS (ESI): [M+H⁺] 354.79

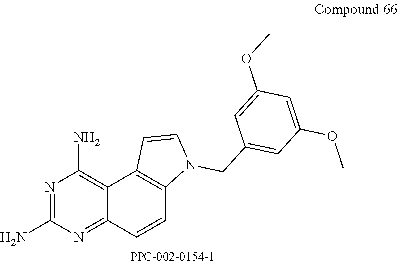 [SMILES: COc1cc(Cn2ccc3c4c(N)nc(N)nc4ccc32)cc(OC)c1]

¹H NMR (400 MHz, DMSO-d₆) δ 7.74 (dd, J=9.0, 0.8 Hz, 1H), 7.62 (d, J=3.2 Hz, 1H), 7.13-7.08 (m, 1H), 7.04 (d, J=9.0 Hz, 1H), 6.75 (s, 2H), 6.38 (t, J=2.4 Hz, 1H), 6.34 (d, J=2.4 Hz, 2H), 5.76 (s, 2H), 5.41 (s, 2H), 3.67 (s, 6H).

MS (ESI): [M+H⁺] 350.34

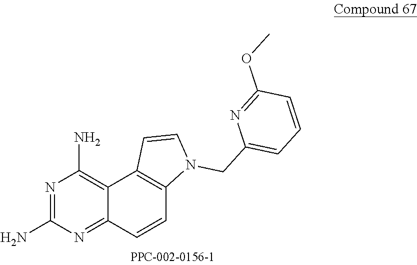 [SMILES: COc1cccc(Cn2ccc3c4c(N)nc(N)nc4ccc32)n1]

¹H NMR (400 MHz, DMSO-d₆) δ 7.79 (dd, J=9.0, 0.8 Hz, 1H), 7.69-7.50 (m, 2H), 7.18-7.09 (m, 1H), 7.05 (d, J=8.8 Hz, 1H), 6.78 (s, 2H), 6.69 (dd, J=8.8, 0.8 Hz, 1H), 6.51 (dd, J=7.2, 0.8 Hz, 1H), 5.80 (s, 2H), 5.49 (s, 2H), 3.82 (s, 3H).

MS (ESI): [M+H⁺] 321.37

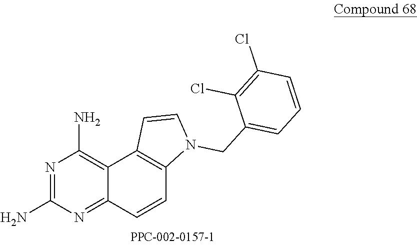 [SMILES: Nc1nc(N)c2c(ccc3c2ccn3Cc2cccc(Cl)c2Cl)n1]

¹H NMR (400 MHz, DMSO-d₆) δ 7.67 (d, J=9.0 Hz, 1H), 7.62-7.51 (m, 2H), 7.33-7.13 (m, 2H), 7.04 (d, J=9.0 Hz, 1H), 6.76 (s, 2H), 6.39 (dd, J=7.6, 1.2 Hz, 1H), 5.76 (s, 2H), 5.65 (s, 2H).

MS (ESI): [M+H⁺] 359.21

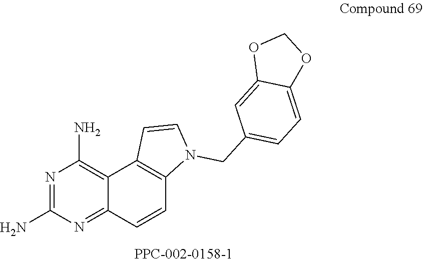 [SMILES: Nc1nc(N)c2c(ccc3c2ccn3Cc2ccc3c(c2)OCO3)n1]

¹H NMR (400 MHz, DMSO-d₆) δ 7.78 (d, J=8.8 Hz, 1H), 7.62 (d, J=3.0 Hz, 1H), 7.08 (d, J=3.0 Hz, 1H), 7.03 (d, J=8.8 Hz, 1H), 6.88-6.79 (m, 2H), 6.76-6.73 (m, 1H), 5.96 (s, 2H), 5.76 (s, 2H), 5.38 (s, 2H).

MS (ESI): [M+H⁺] 334.36

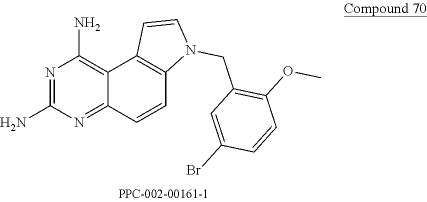 [SMILES: COc1ccc(Br)cc1Cn1ccc2c3c(N)nc(N)nc3ccc21]

¹H NMR (400 MHz, DMSO-d₆) δ 7.71 (d, J=8.8 Hz, 1H), 7.55 (d, J=3.2 Hz, 1H), 7.43 (dd, J=8.8, 2.4 Hz, 1H), 7.10 (d, J=3.2 Hz, 1H), 7.07-7.01 (m, 2H), 6.79 (d, J=2.4 Hz, 1H), 6.73 (s, 2H), 5.75 (s, 2H), 5.42 (s, 2H), 3.87 (s, 3H).

MS (ESI): [M+H+] 398.24.

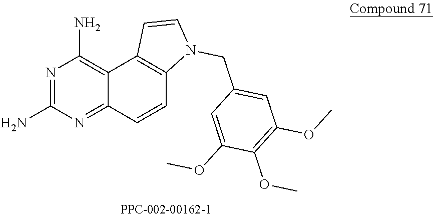 [SMILES: COc1cc(Cn2ccc3c4c(N)nc(N)nc4ccc32)cc(OC)c1OC]

¹H NMR (400 MHz, DMSO-d₆) δ 7.83 (d, J=9.2 Hz, 1H), 7.64 (d, J=3.2 Hz, 1H), 7.09 (d, J=3.2 Hz, 1H), 7.04 (d, J=9.2 Hz, 1H), 6.74 (s, 2H), 6.61 (s, 2H), 5.76 (s, 2H), 5.38 (s, 2H), 3.68 (s, 6H), 3.59 (s, 3H).

MS (ESI): [M+H+] 398.24

Compounds 1-71 were subsequently tested to determine MICs of the compounds relative to the following five species/strains of bacteria: Staphylococcus aureus, NRS384; Enterococcus faecalis, ATCC 51575; Salmonella typhimurium, CMCC 50115; Escherichia coli, ATCC BAA-198; and Acinetobacter baumannii, ATCC 17978. MIC testing of compounds was conducted according to the following protocol.

### 1. Materials and Reagents

| Labware | Supplier | Catalog#/Lot# |
|---|---|---|
| Biological Safety Cabinet | AIRTECH | BSC-1604IIA2 |
| Incubator | Thermo | 371 |
| 96 well V-bottom plates | Axygen | WIPP02280 |
| 96 well round bottom plates | Corning | 3788 |
| Mueller Hinton II Broth (Cation- | BD | 212322 |
| Adjusted)/CAMHB | | |
| Mueller Hinton II Agar (Cation- | BD | 211438 |
| Adjusted)/CAMHA | | |
| Dimethyl sulfoxide(DMSO) | SIGMA | SIGMA-276855-1L |
| NaCl | Richjoint | 20151007 |
| | chemical | |
| Cell densitymeter | Biochrom | Ultrospec 10 |

### 2. Bacterial Strain Panel

Organisms for the assay

| No. | Strain | Strain description |
|---|---|---|
| NRS 384 | Staphylococcus aureus | ATCC |
| ATCC 51575 | Enterococcus faecalis | ATCC |
| CMCC 50115 | Salmonella typhimurium | CMCC |
| ATCC BAA-198 | Escherichia coli | ATCC |
| ATCC 17978 | Acinetobacter baumannii | ATCC |

### 2.1. Strain Preparation and Condition

For each strain to be tested, made fresh streaks onto CAMHA from −80° C. glycerol stocks.

### 2.2. Medium Preparation

CAMHB: Dissolved 22 g of the powder per 1 L of purified water. Mixed well. Autoclaved at 121° C. for 10 minutes. Stored at room temperature (RT) for no more than 1 week.
Saline: Dissolved 9 g of the powder per 1 L of purified water. Mixed well. Autoclaved at 121° C. for 30 minutes. Stored at room temperature (RT) for no more than 1 week.

### 2.3. Preparation of Compound Plates

### 2.3.1. Preparation of Stock Solutions

Prepared the stock solutions of the tested compounds.

### 2.3.2. Preparation of Mother Plate (96-V Bottom Plates)

For compounds:
a) Dispensed 20 ul compound to the wells in column 1, and 20 ul DMSO to the wells in column 2 to 12;
b) Diluted the compounds by transferring 10 ul compounds from column 1 to 20 ul DMSO in column 2; mix with pipette.
c) Repeated until column 11 to get 3×serial dilutions. Column 12 was the DMSO control.

### 2.4. Preparation of Daughter Plates (96-U Bottom Plates)

A multichannel pipette was used to deliver 1 μl of the diluted compound into each well of the corresponding daughter plate.

### 2.5. Preparation of the Inoculum

Scraped 4-8 single colonies from an agar plate into 5 ml saline in a 14 ml Falcon conical tube. Vortexed the tube to suspend the bacteria. Adjusted the turbidity to ˜0.2 (corresponding to 0.5 McFarland) with an Ultrospec 10 Cell density meter.

### 2.6. Addition of Bacteria

The strain suspension was diluted 200-fold in CAMHB medium and dispense into a sterile reservoir. A multichannel pipette was then used to deliver 99 μl of the diluted inocula into each well of the corresponding daughter plate.

### 2.7. Incubation

Incubate the daughter plates at 37° C., 85% humidity for 18-20 hr.

### 2.8. Scoring of MICs

The MIC was read and recorded as the lowest concentration of each agent that completely inhibits visible growth of the microorganism after incubation. A magnifying mirror device was used for ease of scoring the presence or absence of growth in the wells. 96-well micro-plates were photographed.

Results of the MIC testing are provided in Table IV.

TABLE IV
Compound MICs (μg/ml)

| | Staphylococcus | Enterococcus | Salmonella | Escherichia | Acinetobacter |
|---|---|---|---|---|---|
| | aureus, | faecalis, | typhimurium, | coli, ATCC | baumannii, |
| Compound | NRS384 | ATCC 51575 | CMCC 50115 | BAA-198 | ATCC 17978 |
| 1 | 0.088 | 0.01 | 0.088 | 2.37 | 2.37 |
| 2 | 2.37 | 2.37 | >64 | 64 | 7.11 |
| 3 | 0.26 | 0.03 | 0.79 | 7.11 | 7.11 |
| 4 | 0.03 | 0.03 | 0.79 | 7.11 | 7.11 |
| 5 | 0.26 | 0.03 | 0.79 | 7.11 | 7.11 |
| 6 | 0.26 | 0.03 | 0.79 | 7.11 | 7.11 |
| 7 | >64 | >64 | >64 | >64 | >64 |
| 8 | 0.088 | 0.03 | 0.26 | 2.37 | 0.79 |
| 9 | 0.79 | 0.26 | 2.37 | 21.3 | 21.3 |
| 10 | 0.088 | 0.01 | 0.088 | 2.37 | 0.79 |
| 11 | 0.088 | 0.03 | 0.79 | 21.3 | 7.11 |
| 12 | 0.26 | 0.03 | 0.79 | 7.11 | 2.37 |
| 13 | 0.088 | 0.01 | 0.088 | 7.11 | 2.37 |
| 14 | 0.088 | 0.01 | 0.26 | 7.11 | 2.37 |
| 15 | 0.26 | 0.09 | 2.37 | 7.11 | 21.3 |
| 16 | 0.79 | 0.09 | 21.3 | >64 | >64 |
| 17 | 2.37 | 0.79 | 7.11 | 21.3 | 21.3 |
| 18 | 2.37 | 0.79 | 7.11 | 21.3 | 21.3 |
| 19 | 2.37 | 0.79 | 21.3 | >64 | >64 |
| 20 | 2.37 | 0.79 | 21.3 | >64 | 64 |
| 21 | 0.26 | 0.09 | 0.79 | 7.11 | 21.3 |
| 22 | 0.79 | 0.09 | 7.11 | 21.3 | >64 |
| 23 | 2.37 | 0.26 | >64 | >64 | >64 |
| 24 | 0.79 | 0.26 | 0.26 | 7.11 | 64 |
| 25 | 0.26 | 0.09 | 0.79 | 7.11 | 21.3 |
| 26 | 0.09 | 0.09 | 0.26 | 2.37 | 2.37 |
| 27 | >64 | >64 | >64 | >64 | >64 |
| 28 | 21.3 | 7.11 | >64 | >64 | >64 |
| 29 | 64 | 21.3 | >64 | >64 | >64 |
| 30 | 7.11 | 2.37 | >64 | >64 | >64 |
| 31 | 0.26 | 0.09 | 0.79 | 7.11 | 7.11 |
| 32 | 0.09 | 0.09 | 0.09 | 7.11 | 21.33 |
| 33 | 0.26 | 0.03 | 0.26 | 2.37 | 7.11 |
| 34 | 0.26 | 0.09 | 0.26 | 7.11 | 21.3 |
| 35 | 2.37 | 0.26 | 0.26 | 21.3 | 7.11 |
| 36 | 0.26 | 0.03 | 0.79 | 21.3 | 7.11 |
| 37 | 0.03 | 0.01 | 0.26 | 2.37 | 2.37 |
| 38 | 0.03 | 0.003 | 0.26 | 2.37 | 2.37 |
| 39 | 0.79 | 0.09 | 2.37 | 7.11 | 21.3 |
| 40 | 0.26 | 0.09 | 0.26 | 7.11 | 2.37 |
| 41 | 2.37 | 0.09 | 0.79 | 64 | 21.3 |
| 42 | 0.03 | 0.01 | 0.79 | 7.11 | 7.11 |
| 43 | 2.37 | 0.09 | 2.37 | 64 | 64 |
| 44 | 0.09 | 0.03 | 7.11 | 7.11 | 64 |
| 45 | 0.26 | 0.01 | 0.003 | 7.11 | 2.37 |
| 46 | 0.79 | ≤0.001 | 2.37 | >64 | 7.11 |
| 47 | 0.09 | 0.003 | 2.37 | >64 | >64 |
| 48 | 0.09 | ≤0.001 | 0.003 | 7.11 | 0.79 |
| 49 | 2.37 | 0.26 | 2.37 | >64 | >64 |
| 50 | 0.09 | 0.003 | 0.03 | 7.11 | 7.11 |
| 51 | 0.26 | 0.003 | 0.26 | 21.3 | >64 |
| 52 | 7.11 | 2.37 | 21.3 | >64 | >64 |
| 53 | 0.26 | 0.26 | 0.26 | 21.3 | 64 |
| 54 | 2.37 | 0.003 | 21.3 | >64 | 64 |
| 55 | 0.26 | 0.03 | 0.26 | 7.11 | 2.37 |
| 56 | 0.03 | 0.01 | 0.03 | 7.11 | 2.37 |
| 57 | 0.09 | 0.03 | 0.09 | 7.11 | 7.11 |
| 58 | 0.09 | 0.03 | 0.26 | 7.11 | 2.37 |
| 59 | 0.26 | 0.03 | 0.26 | 21.3 | 7.11 |
| 60 | 0.79 | 0.09 | 0.79 | 7.11 | 21.3 |
| 61 | 0.09 | 0.03 | 0.26 | 7.11 | 7.11 |
| 62 | 0.79 | 0.003 | 0.26 | 21.3 | 21.3 |
| 63 | 0.26 | 0.09 | 0.26 | 21.3 | 21.3 |
| 64 | 7.11 | 21.3 | 64 | 64 | >64 |
| 65 | 0.09 | 0.01 | 0.26 | 7.11 | 7.11 |
| 66 | 0.09 | 0.01 | 0.26 | 2.37 | 7.11 |
| 67 | 0.09 | 0.01 | 0.26 | 2.37 | 0.26 |
| 68 | 0.03 | 0.01 | 0.26 | 2.37 | 2.37 |
| 69 | 0.09 | 0.03 | 0.26 | 2.37 | 7.11 |
| 70 | 0.01 | ≤0.001 | 0.26 | 2.37 | 2.37 |
| 71 | 0.03 | 0.01 | 0.79 | 2.37 | 7.11 |

The MIC of compounds 10, 14, 32, 40, 45, and 48 against P. aeruginosa PA14 were also determined according to the protocol above. Table V provides the results.

TABLE V
MICs against P. aeruginosa PA14 (μg/ml)

| Compound | P. aeruginosa PA14 |
|---|---|
| 10 | 25 |
| 14 | 25 |
| 32 | 8.8 |
| 40 | 45 |
| 45 | 6.3 |
| 48 | 12.5 |

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A compound of Formula (I) and/or salts thereof: 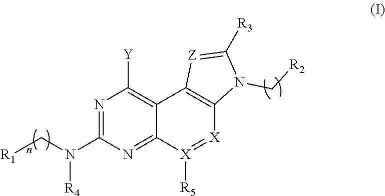
wherein R₁, R₃, R₄ and R₅ are independently selected from the group consisting of hydrogen, alkyl, alkenyl, heteroalkyl, cycloalkyl, heterocycloalkyl, aryl, heteroaryl, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of arylene-alkynyl, heteroarylene-alkynyl, arylene-alkenyl, heteroarylene-alkenyl, alkylnylene-alkyl, alkynylene-cycloalkyl, alkynylene-heterocycloalkyl, alkynylene-aryl, alkynylene-heteroaryl, alkenylene-aryl, alkenylene-heteroaryl, alkynylene-amine, alkynylene-protected amine, and alkynylene-alkylsilane; and
wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and
wherein Y is selected from the group consisting of OH and NR₁₂R₁₃, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure; and n is an integer from 0 to 5.

2. The compound of claim 1, wherein R₂ is selected from the group consisting of arylene-alkynyl, heteroarylene-alkynyl, alkylnylene-alkyl, alkynylene-cycloalkyl, alkynylene-heterocycloalkyl, alkynylene-aryl, and alkynylene-heteroaryl.

3. The compound of claim 2, wherein R₁ and R₄ are independently selected from the group consisting of hydrogen, alkyl, cycloalkyl, and heterocycloalkyl.

4. The compound of claim 2, wherein Y is NR₁₂R₁₃, wherein R₁₂ and R₁₃ are independently selected from the group consisting of hydrogen, alkyl, heteroalkyl, cycloalkyl, heterocycloalkyl, alkenyl, aryl, and heteroaryl.

5. The compound of claim 2, wherein X and Z are independently selected from C and N.

6. The compound of claim 2, wherein X and Z are each C.

7. The compound of claim 2, wherein R₃ is selected from the group consisting of hydrogen and alkyl.

8. The compound of claim 2 having the formula: 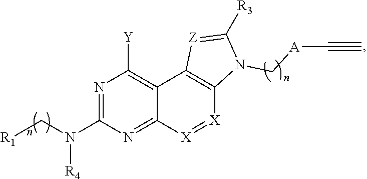
wherein A is selected from the group consisting of aryl and heteroaryl.

9. The compound of claim 8, wherein X and Z are selected from the group consisting of C and N, and wherein R₁, R₃, and R₄ are independently selected from the group consisting of hydrogen, alkyl, cycloalkyl, and heterocycloalkyl.

10. A pharmaceutical composition comprising a compound of Formula (I) and/or a salt thereof: 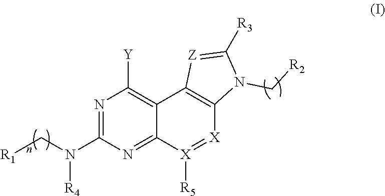
wherein R₁, R₃, R₄ and R₅ are independently selected from the group consisting of hydrogen, alkyl, alkenyl, heteroalkyl, cycloalkyl, heterocycloalkyl, aryl, heteroaryl, alkylene-aryl, alkylene-heteroaryl, amide, sulfonamide, acid, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of arylene-alkynyl, heteroarylene-alkynyl, arylene-alkenyl, heteroarylene-alkenyl, alkylnylene-alkyl, alkynylene-cycloalkyl, alkynylene-heterocycloalkyl, alkynylene-aryl, alkynylene-heteroaryl, alkenylene-aryl, alkenylene-heteroaryl, alkynylene-amine, alkynylene-protected amine, and alkynylene-alkylsilane; and
wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and
wherein Y is selected from the group consisting of OH and NR₁₂R₁₃, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure; and n is an integer from 0 to 5,
wherein the compound of Formula (I) is present in the pharmaceutical composition in an amount sufficient to exhibit antibacterial properties.

11. The pharmaceutical composition of claim 10, wherein the compound of Formula (I) is present at a minimum inhibitory concentration of 0.0005 μg/ml to 1 mg/ml for bacterial growth.

12. The pharmaceutical composition of claim 10, wherein the compound of Formula (I) is present at a minimum inhibitory concentration of 0.001 μg/ml to 100 μg/ml for bacterial growth.

13. The pharmaceutical composition of claim 10, wherein the compound of Formula (I) is present at a minimum inhibitory concentration of 0.001 μg/ml to 10 μg/ml for bacterial growth.

14. The pharmaceutical compositions of claim 10, wherein R₂ is selected from the group consisting of arylene-alkynyl, heteroarylene-alkynyl, alkylnylene-alkyl, alkynylene-cycloalkyl, alkynylene-heterocycloalkyl, alkynylene-aryl, and alkynylene-heteroaryl.

15. The pharmaceutical compositions of claim 14, wherein R₁ and R₄ are independently selected from the group consisting of hydrogen, alkyl, cycloalkyl, and heterocycloalkyl.

16. The pharmaceutical compositions of claim 14, wherein Y is NR₁₂R₁₃, wherein R₁₂ and R₁₃ are independently selected from the group consisting of hydrogen, alkyl, heteroalkyl, cycloalkyl, heterocycloalkyl, alkenyl, aryl, and heteroaryl.

17. The pharmaceutical compositions of claim 14, wherein X and Z are independently selected from C and N.

18. The pharmaceutical composition of claim 14, wherein X and Z are each C.

19. The pharmaceutical composition of claim 10, wherein the compound is of the formula” 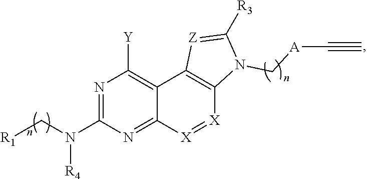
wherein A is selected from the group consisting of aryl and heteroaryl.

20. The pharmaceutical composition of claim 19, wherein X and Z are selected from the group consisting of C and N, and wherein R₁, R₃, and R₄ are independently selected from the group consisting of hydrogen, alkyl, cycloalkyl, and heterocycloalkyl.

21. The pharmaceutical composition of claim 10 exhibiting antibacterial properties against gram negative bacteria.

22. The pharmaceutical composition of claim 12, wherein the bacterial growth is that of P. aeruginosa.
